(19) United States
(12) Patent Application Publication
(10) Pub. No.: US 2025/0176807 A1
(43) Pub. Date: Jun. 5, 2025

(54) ENDOSCOPE INSERTION TUBE WITH GRADED STATIC STIFFNESS

(71) Applicant: AdaptivEndo LLC, Louisville, KY (US)

(72) Inventors: Galen R. Powers, Louisville, KY (US); Mark A. Griffin, Louisville, KY (US); Walter Stubblefield, Louisville, KY (US); Hershel E. Fancher, Georgetown, IN (US)

(21) Appl. No.: 19/046,119

(22) Filed: Feb. 5, 2025

Related U.S. Application Data
(63) Continuation of application No. PCT/US2023/029581, filed on Aug. 5, 2023.
(60) Provisional application No. 63/370,538, filed on Aug. 5, 2022.

(51) Int. Cl. A61B 1/005; A61B 1/00

(52) U.S. Cl. CPC A61B 1/0055; A61B 1/0011

(57) ABSTRACT

An endoscope is provided. The endoscope incorporates a single-use shaft assembly having an insertion tube with a distal articulating section. The insertion tube comprises an outer coil, two or more pull wires, each located with a concentric compression coil, an outer braid, an outer sheath, and a stiffening element. The distal articulating section comprises a deformable element for articulating the distal tip. The stiffening element may be made with material with a uniform elasticity or multiple materials with differing elasticity along its length. The stiffening element may further include a cross-sectional geometry along the length of the stiffening element. The cross-sectional geometry may be uniform along the length or differ along the length for graded stiffness along the length of the insertion tube. Methods of manufacturing are also disclosed.

### CROSS-REFERENCE TO RELATED APPLICATION

This application claims the benefit of International Application No. PCT/US2023/029581 filed Aug. 5, 2023, which claims the benefit of U.S. Provisional Application No. 63/370,538 filed Aug. 5, 2022 the entire disclosures of which are hereby incorporated by reference.

### BACKGROUND OF THE DISCLOSURE

This disclosure generally relates to endoscopes.

An endoscope insertion tube is typically a hand assembled composite incorporating a coil, braid, and one or more outer extrusions (one or more different durometer polymers) that is then reflowed into composite adhered together by the reflowed outer polymeric jacket. Alternatively, a braided coil can be fed through an extruder with one or more extrusion resin heads (different durometer resins). Alternative configurations include varying the braid pattern and coil pattern. The goal of these variations along the length of the insertion tube is to obtain varying stiffness down the length of the insertion tube. For instance, when traversing the gastric cavity with either a duodenoscope or a gastroscope, increased stiffness in specific sections of the insertion tube assist in preventing looping within the gastric cavity. Similarly, varying the stiffness along the length of a colonoscope can assist in navigating along the elastic walls of the colon while avoiding unwanted trauma. This process is generally fairly expensive and a lower cost alternative is desirable.

Thus, there is a need for improvement in this field.

### SUMMARY OF THE DISCLOSURE

Disclosed are single-use shaft assemblies for a medical device, for example an endoscope. The single-use shaft assembly may be configured for use with a reusable hand-piece assembly having one or more controls for manipulating the single-use shaft assembly. The single-use shaft assembly may include disposable flexible endoscope shaft having an insertion tube assembly. The insertion tube assembly may include an outer coil with a length extending along at least a portion of the length of the insertion tube assembly. The insertion tube assembly may further include a braided sleeve surrounding at least a portion of the length of the outer coil. An outer sheath may be used to surround the braided sleeve and outer coil. The insertion tube assembly may further include a stiffening element extending along at least a portion of the length of the insertion tube assembly.

Disposable flexible endoscope shafts of the present disclosure comprise a proximal portion having an insertion tube assembly and a distal portion having an articulating section assembly.

Insertion tube assemblies disclosed herein include an outer coil. Positioned within the outer coil are a plurality of compression coils. The compression coils may be arranged to increase the column strength of the outer coil along a longitudinal direction. The compression coils can be helically wound metal wires and/or spiral cut cannulas that provide for lateral flexibility. Articulation wires can be slidably positioned within lumens defined by the compression coils.

The insertion tube assembly can include a sleeve (e.g., braided sleeve) positioned around the outer coil. The sleeve preferably provides torsional strength to the insertion tube assembly. The braided sleeve can include a metal braid or plastic braids such as PET.

The insertion tube assembly comprises an outer sheath positioned around sleeve. The outer sheath may be applied as a reflowed tube or by an extruder. The outer sheath can bond to the sleeve and/or to the outer coil through apertures of the sleeve.

The articulating section assembly comprises an articulating section having a plurality of hinges. Each hinge provides rotation around a pivot axis. The pivot axes of hinges can extend transverse to a longitudinal axis of the articulating section. Additionally, the pivot axis of one or more of the hinges can be in a different plane than the pivot axis of one or more other hinges. For example, the pivot axes of hinges can be in alternatively located in planes perpendicular to one another when the articulating section is in a straight (e.g., unbent) configuration. Advantageously, such an arrangement can provide an articulating element capable of articulating the distal tip/camera in three dimensions.

One or more hinges of the articulating section can be living hinges. In some instances, the articulating section is a unitary articulating section form. The unitary articulating section can be formed from a single piece of material. The unitary articulating section structure can be fabricated using injection molding or additive material fabrication techniques. Alternatively, the unitary articulating section can be formed by extruding a cylinder and cutting the cylinder tube with a knife, laser, milling tool, water jet, or other material removal mechanism to form the living hinges. As will be appreciated, the bending and torque fidelity characteristics of the articulating section can be configured by configuring the angles of the cuts/recesses that define the hinges and/or the distance between adjacent hinges.

In another arrangement, the articulating section structure comprises a plurality of discrete links that, when assembled, define a plurality of concentric tab and socket pivot joints that function as a hinge. As mentioned above, each hinge (e.g., tab and socket pivot join) can provide for rotation around a pivot axis in a single plane. Moreover, the plurality of concentric tab and socket joints can be alternatingly located in two perpendicular planes when the central axis of all links are aligned so as to provide the articulating section with multiple degrees of freedom.

An outer sheath can be positioned around the articulating section to prevent contaminants from entering the one or more hinges and/or lumens defined by the articulating section. The articulating section can include a distal cap defining an air/water nozzle, an instrument tube outlet, a camera outlet, and/or an LED outlet.

The insertion tube assembly and articulating section can be bonded together (e.g., heat or friction welding, adhesive, etc.) and/or attached together with mating features on the contacting surface (e.g., threads) or with a transition tube, as shown in the illustrated embodiment. The mid-plane of the transition tube can be located at the transition between the insertion tube assembly and the articulating section, and the transition tube can be bonded (e.g., swaged or adhered with adhesive) onto both the insertion tube assembly and the distal articulating section to form a secure attachment. The transition tube can be deformable to allow deflection of the flexible endoscope shaft at the transition.

The insertion tube assemblies disclosed herein can be manufactured using a continuous (e.g., reel-to-reel) manufacturing process. The braided sleeve can be applied around the outer coil during the continuous manufacturing process. Additionally, the outer sheath can be applied during the continuous manufacturing process. For example, the assembly of the outer coil and surrounding braided sleeve can pass through one or more extrusion heads during the continuous manufacturing process so as to apply the outer sheath to the portion of the insertion tube assembly. Such a process can create a smooth outer sheath that is integrally bonded to the outer coil and/or braided sleeve. The outer sheath may have a varying durometer along a length of the shaft.

After positioning of the outer sheath around the assembly, the shaft may be cut to the desired length and compression coils and/or articulation wires inserted into an interior of the outer coil.

Advantageously, providing continuous manufacturing of an insertion tube assembly can reduce the cost of manufacturing the endoscope shaft assembly and increase production speed. Accordingly, in certain aspects, the present disclosure provides a low-cost, flexible endoscope shaft and method of manufacturing same. As the insertion tube assembly can be manufactured continuously, desired lengths of insertion tube assembly, or a portion thereof, can be cut to length after the outer sheath extrusion process or cut from a finish goods reel. Advantageously, continuous techniques for fabricating the insertion tube (e.g., reel-to-reel techniques) avoid braiding and coating the insertion tube in discrete sections using labor intensive processes.

As will be appreciated, the insertion tube assemblies disclosed can provide containment of wiring, tubes, and actuation wires of the endoscope shaft while having torsional and compressive strength sufficient to advance the articulating section assembly through tortuous vessels of a patient.

Methods of fabricating at least a portion of an insertion tube assembly are disclosed. Said methods may comprise forming a continuous first length, second length, and third length of outer coil using a continuous wire coiler. Forming the first length defines a first period of time. Forming the second length defines a second period of time. Forming the third length defines a third period of time. An outer braid is formed around the outer coil along the first length during the second period of time to make a braided assembly. An outer sheath may be extruded over the braded assembly along the first length during the third period of time to form at least a portion of an insertion tube. A stiffening element may be inserted within the insertion tube along at least a portion of a length of the insertion tube assembly before or after the insertion tube is cut to length.

In some embodiments, the stiffening element may extend along substantially the entire length of the insertion tube.

In some examples, the stiffening element may be fixed to a portion of an endoscope handle (e.g., to a housing of a single-use shaft assembly). In other embodiments, the stiffening element may be fixed to a proximal end of the insertion tube. The stiffening element may be fixed by a friction fit, an interference fit, retention geometry (e.g., threaded interface), and/or an adhesive.

In some embodiments, a distal end of the stiffening element is freely moveable within the insertion tube. In other examples, the distal end of the stiffening element is fixed at one or more locations along a length of the insertion tube. In yet other embodiments, the distal end of the stiffening element is fixed to a distal portion of the insertion tube.

The stiffening element may be comprised of a uniform material with the same elasticity and cross-sectional geometry along substantially the entire length of the stiffening element within the endoscope. The stiffening element may extend along a length of the insertion tube. The stiffening element may terminate prior to a distal end of the insertion tube.

In other embodiments, the stiffening element may include a first length and a second length. The stiffening element may be composed of a uniform material with the same elasticity along the first length and the second length of the stiffening element. The stiffening may include a first cross-sectional geometry along the first length and a second cross-sectional geometry along the second length. The first cross-sectional geometry may be different than the second-cross-sectional geometry.

In yet another embodiment, the stiffening element may be comprised of a uniform material with the same elasticity and cross-sectional geometry along substantially the entire length of the stiffening element. The cross-sectional geometry may be asymmetrical.

In another example, the stiffening element may include a first length and a second length, wherein the first length may be comprised of a first material and the second length may be comprised of a second material. The first material may have a modulus of elasticity different than a modulus of elasticity of the second material.

The inventive aspects and embodiments discussed below in the following separate paragraphs of the summary may be used independently or in combination with each other.

Further forms, objects, features, aspects, benefits, advantages, and embodiments of the present disclosure will become apparent from a detailed description and drawings provided herewith.

### BRIEF DESCRIPTION OF THE DRAWINGS

FIG. 1 illustrates a perspective view of a disposable flexible endoscope shaft assembly.

FIG. 2A illustrates a perspective view of an insertion tube assembly.

FIG. 2B illustrates a perspective view of a distal end of the insertion tube assembly.

FIG. 3 illustrates an exploded view of the insertion tube assembly.

FIG. 4 illustrates a perspective view of the articulating section assembly.

FIG. 5A illustrates an exploded view of the articulating section assembly.

FIG. 5B is a partial exploded view of the articulating section assembly.

FIG. 6A illustrates a perspective view of the unitary articulating section in a straight configuration.

FIG. 6B illustrates a perspective view of the unitary articulating section in a deflected configuration.

FIG. 6C illustrates a close-up view of the unitary articulating section.

FIG. 7A illustrates a close-up of the proximal end of the unitary articulating section of FIGS. 6A-6C.

FIG. 7B illustrates a close-up of the distal end of the unitary articulating section of FIGS. 6A-7A.

FIG. 8 illustrates a perspective view of an articulating link assembly.

FIG. 9A illustrates a perspective view of an articulating link.

FIG. 9B illustrates a perspective view of the proximal articulating link.

FIG. 9C illustrates a perspective view of the distal articulating link.

FIG. 10 illustrates a perspective view of the articulation pull wire and termination ring assembly.

FIG. 11 is a flowchart illustrating a process for manufacturing disposable flexible endoscope shafts disclosed herein.

FIG. 12 illustrates a first portion of a manufacturing arrangement.

FIG. 13 illustrates a second portion of a manufacturing arrangement.

FIG. 14 illustrates a perspective view of an endoscope.

FIG. 15 illustrates a perspective view of a distal end of the insertion tube.

FIG. 16 illustrates a perspective view of a distal end of the insertion tube with various tubing and wires extending through and from the insertion tube.

FIG. 17 illustrates a cross-sectional view of an insertion tube with a stiffening element extending along substantially the entire length of the insertion tube.

FIG. 18 illustrates a cross-sectional view of an insertion tube with a stiffening element extending along a portion of the length of the insertion tube.

FIG. 19 illustrates a cross-sectional view of an insertion tube with a tapered stiffening element.

FIG. 20A-E illustrates a cross-sectional view of cross-sectional geometry options for stiffening element: A) solid round B) hollow round C) octagonal D) square E) T-shaped.

FIG. 21 illustrates a cross-sectional view of an insertion tube with a stiffening element composed of one or more materials of differing modulus of elasticities along the length.

### DETAILED DESCRIPTION OF THE DISCLOSURE

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

As used herein, “proximal” refers to an end or direction associated with a physician or other treating personnel during a device operation, and “distal” refers to the opposite end (“patient end/treating end”). The drawing figures referred to herein are provided for illustrative purposes only. They should not be construed as limiting the scope of the disclosure defined by the claims, including that they may not necessarily be drawn to scale.

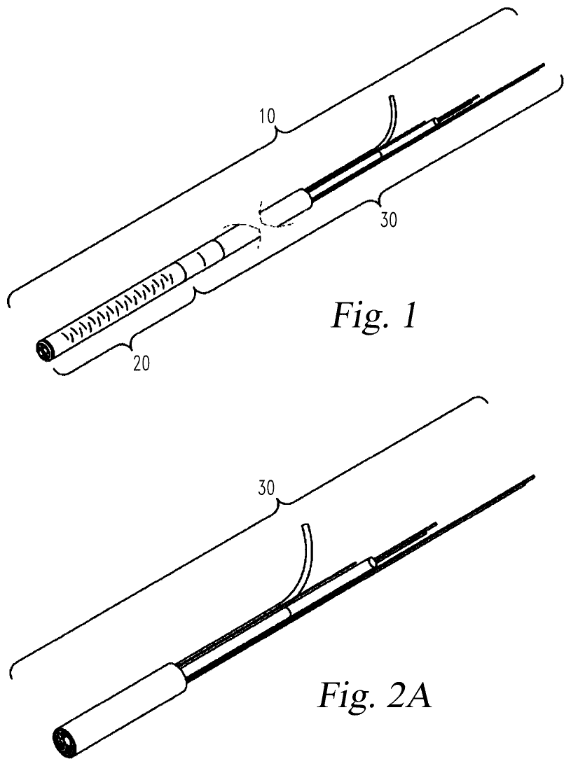

FIG. 1 illustrates a disposable flexible endoscope shaft assembly 10 having a distal portion comprising an articulating section assembly 20 and a proximal portion comprising an insertion tube assembly 30.

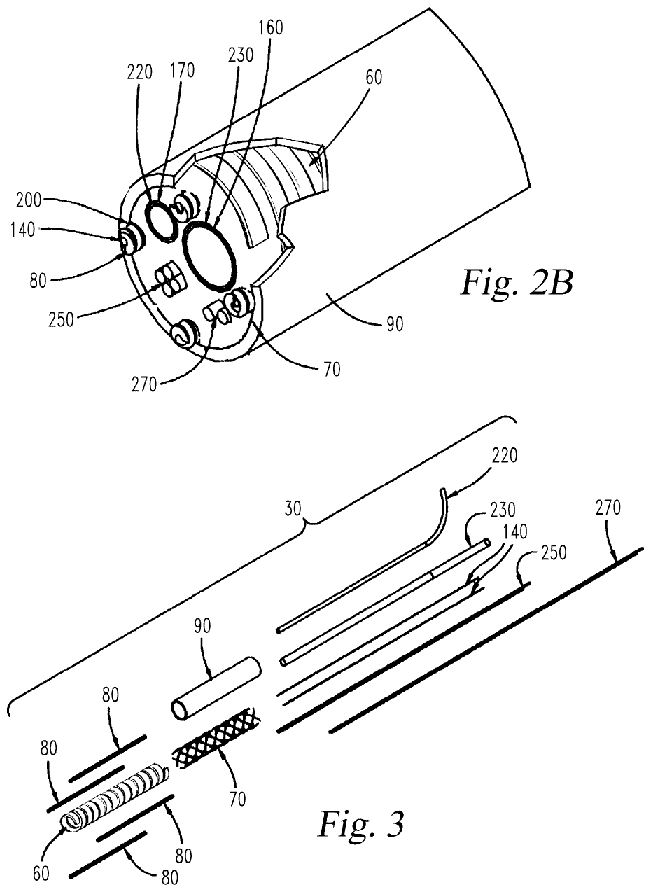

Turning now to FIGS. 2A, 2B and 3, the insertion tube assembly 30 includes an outer coil 60. An articulation wire 140 and a compression coil 80 extend along a length of outer coil 60 through its interior.

A braided sleeve 70 surrounds the outer coil 60 and is positioned between the outer coil and an outer sheath 90. The outer sheath 90 can be applied over the braided sleeve 70 and outer coil 60 as a reflowed tube (e.g., thermal lamination) or by extrusion.

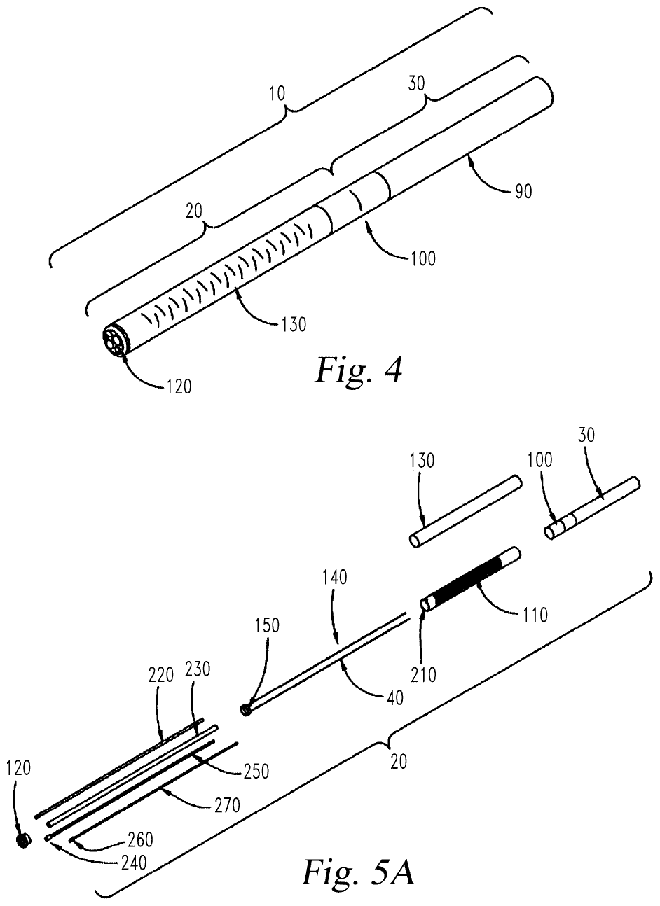

FIG. 4 illustrates a cap 120 at the distal end of the articulating section 20 and an articulating section outer sheath 130. The articulating section assembly 20 is connected to the insertion tube assembly 30 with a transition tube 100. The transition tube 100 may be mechanically secured to both the articulating section assembly 20 and the insertion tube assembly 30 through a process such as swaging.

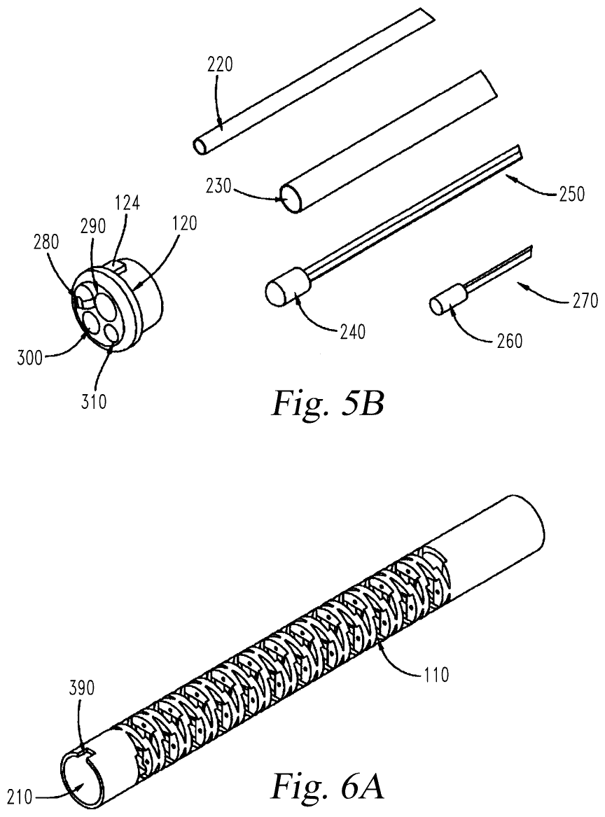

FIGS. 5A and 5B depict the articulating section assembly 20. The articulating section assembly 20 comprises an articulation wire and termination ring assembly 40 having a termination ring 150 positioned at a distal end of articulation wires 140, a unitary articulating section 110, an articulating section outer sheath 130, cap 120, an instrument tube 230, an air/water tube 220, a camera 240 and camera wiring harness 250, a light emitter 260 (e.g., an LED) and light emitter wiring harness 270. When disposable flexible endoscope shaft assembly 10 is assembled, air/water tube 220 extends through a lumen 210 defined by the articulating section 110, instrument tube 230 extends through lumen 210, camera wiring harness 242 extends through lumen 210, and light emitter wire harness 270 extends through lumen 210.

Cap 120 defines an air/water nozzle 280, an instrument tube outlet 290, a camera outlet 300, and a light emitter outlet 310. Cap 120 includes a cap alignment tab 124 arranged to engage a cap alignment notch of an articulating section (e.g., unitary articulating section 110).

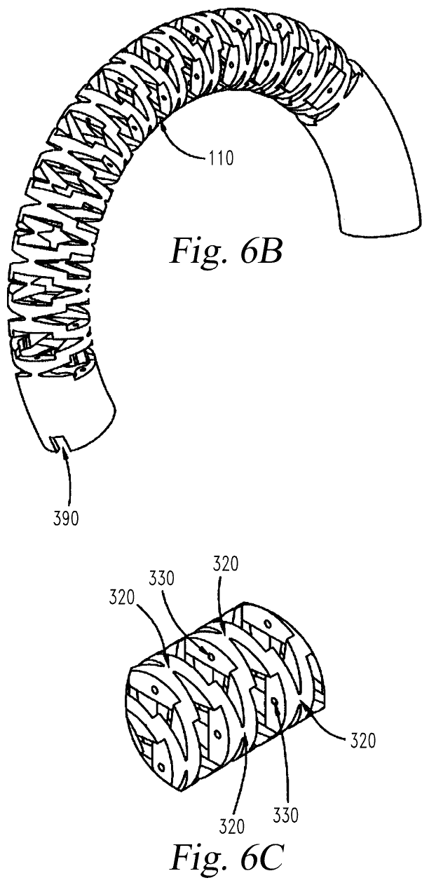

FIGS. 6A-6C depict the unitary articulating section 110 comprising a plurality of living hinges 320 being alternatingly located in two perpendicular planes when unitary articulating section is in a straight configuration. Each deformable living hinge element 320 provides means for rotation around a pivot axis in a single plane. An articulation wire lumen 330 traverses each living hinge element 320 and is arranged to receive an articulation wire 140.

The lumen 210 located within the unitary articulating section 110 can receive an air/water tube, an instrument tube, and/or wiring 210. A cap alignment notch 390 is located at the distal end of the unitary articulating section 110 and arranged to receive the cap alignment tab 124.

FIG. 6B illustrates the unitary articulating section 110 in a deformed configuration consistent with an articulation of 180° for retrograde viewing using the distally mounted camera 240.

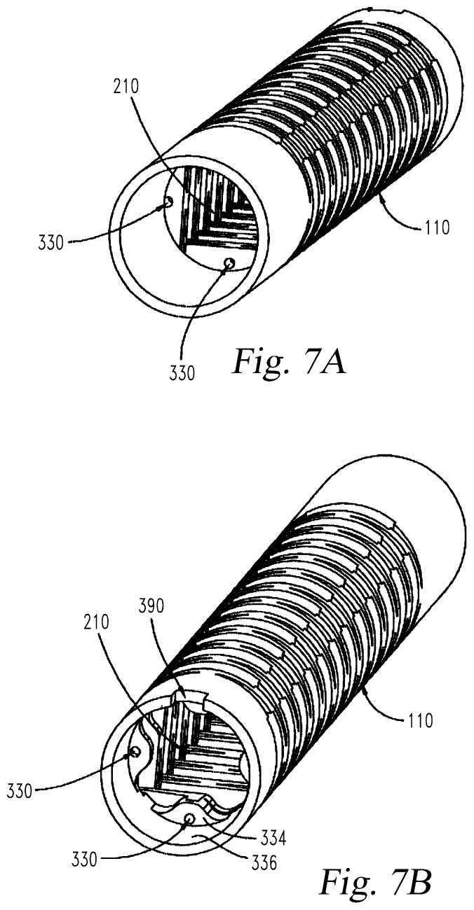

FIG. 7A illustrates the proximal end of the unitary articulating section 110, and FIG. 7B illustrates the distal end of the unitary articulating section 110. As seen in FIG. 7B, a distally facing surface 334 extends inwardly from the inner surface 336 of the unitary articulating section 110. Distally facing surface 334 is arranged to contact termination ring 150 and transfer tensile force from the articulation wire and termination ring assembly 40 to the unitary articulating section 110.

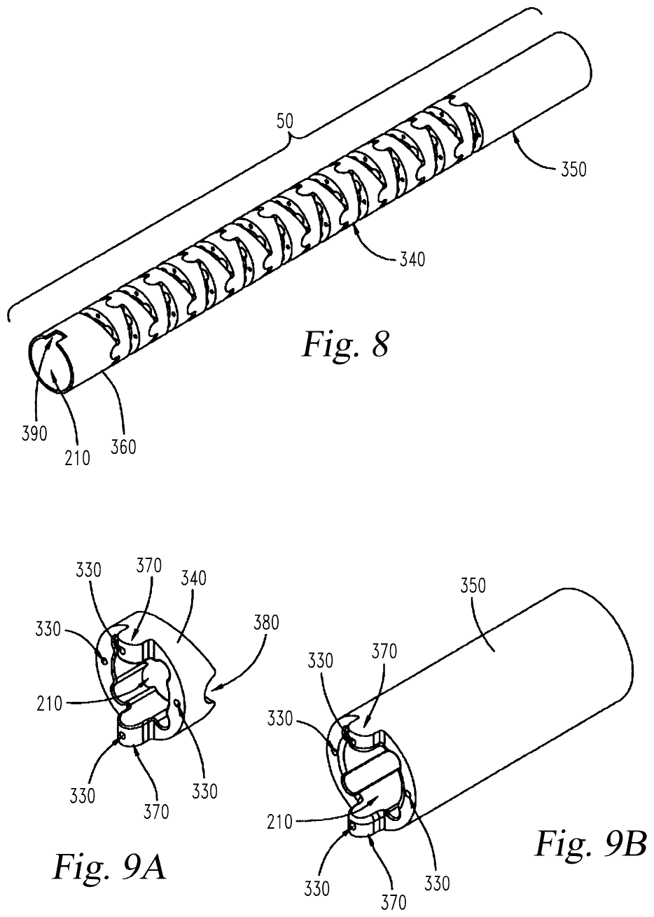

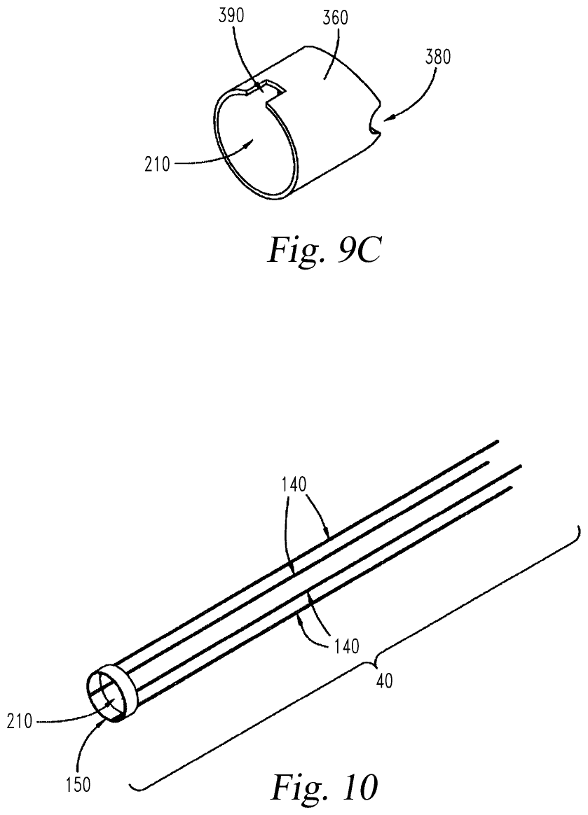

FIGS. 8-9C illustrate an articulating link assembly 50 comprising a proximal articulating link 350, an intermediate articulating link 340, and a distal articulating link 360. The proximal articulating link 350, intermediate articulating link 340, and distal articulating link 360 each define a lumen 210 arranged to receive an air/water tube, an instrument tube, and/or wiring.

The proximal articulating link 350 includes pivot tabs 370 located in a first (e.g., vertical) plane. The intermediate articulating link 340 comprises articulation pull wire lumens 330, pivot tabs 370 located in a first plane, and pivot sockets 380 located in a second plane. The distal articulating link 360 includes a cap alignment notch 390 to control alignment of the camera 240 relative to each of the four articulation pull wires 140 and two pivot sockets 380 located in the first or second plane. When assembled, the pivot tabs 370 are received within and pivotable relative to the pivot sockets 380.

FIG. 10 illustrates the articulation pull wire and termination ring assembly 40 comprising four articulation pull wires 140 and an articulation pull wire termination ring 150. The interior of the articulation pull wire termination ring 150 defines lumen 210 for passage of air/water tube, instrument tube, and wiring.

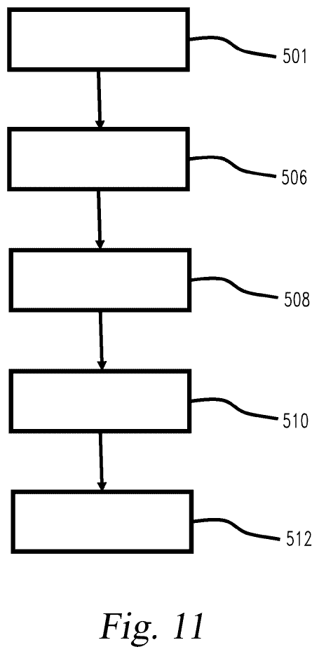

Turning now to FIG. 11, a process for manufacturing insertion tube assemblies disclosed herein is described. The insertion tube assemblies can be manufactured using a continuous manufacturing process (e.g., reel-to-reel). The process can begin in stage 501 with a continuous coiling process wherein the coil is manufactured in a continuous way (e.g., a continuous wire coiler).

In stage 506, the braided sleeve can be applied around the outer coil. This can also occur during a continuous manufacturing process.

In stage 508, the outer sheath can be applied. This, again, can occur during a continuous manufacturing process. For example, the outer coil and braided sleeve assembly can pass through one or more extrusion heads that extrude the outer sheath around the assembly. Such a process can create a smooth outer sheath that is integrally bonded to the outer coil and/or braided sleeve. The outer sheath may have a varying durometer along its length such that some areas (e.g., lengths) of the outer sheath have a greater durometer than other areas. This may be accomplished by extruding resins of different durometers (e.g., different resins) through separate extrusion heads of the one or more extrusion heads or by extruding resins of different durometers through at least one extrusion head of the one or more extrusion heads.

In stage 510, the desired length of insertion tube assembly, or a portion thereof, can be cut to length. This can occur immediately after the outer sheath extrusion process or, for example, from a reel of finished goods. After cutting the assembly to length, articulation wires and/or compression coils may be inserted into the lumen of the outer coil. The process concludes in stage 512.

As will be appreciated by those skilled in the art, this continuous technique for fabricating the insertion tube avoids labor and time intensive batch processes currently used to form discrete sections of endoscope shafts. Applicant believes this can reduce the costs of manufacturing endoscope shaft assemblies and/or increase production speed.

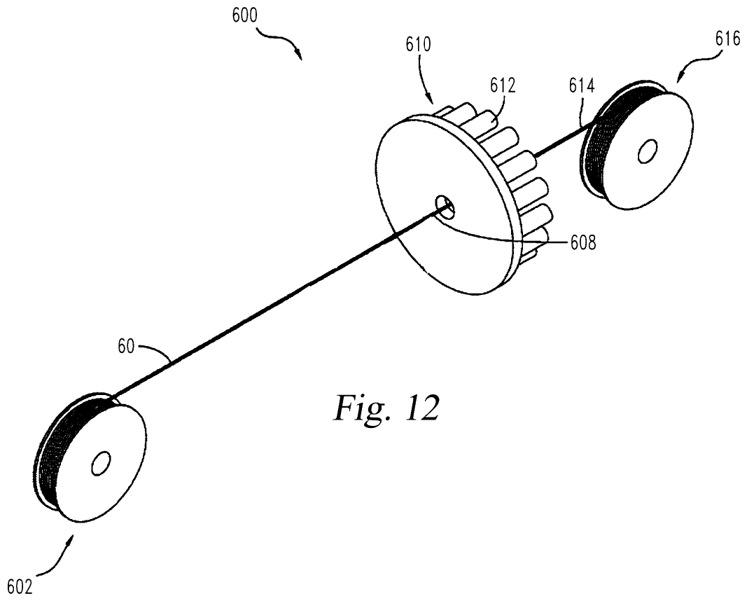

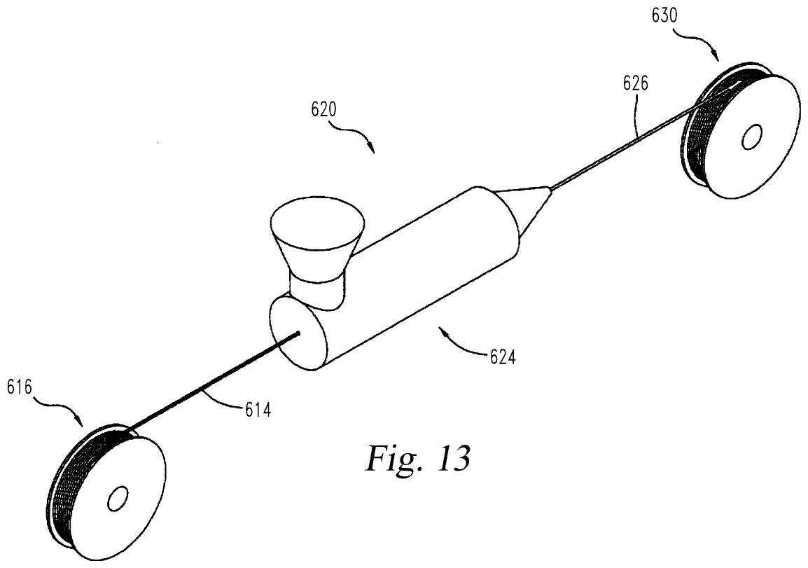

FIGS. 12 and 13 illustrate a manufacturing arrangement suitable for practicing the process described above. In FIG. 12, a first portion 600 of the manufacturing arrangement is shown. First portion 600 includes an outer coil 60 extending from an outer coil reel 602 to fixture 610. Fixture 610 is arranged to form a braided sleeve around the outer coil as the outer coil and compression coil continually advances through fixture 610. For example, fixture 610 may include a braiding machine having a plurality of bobbins 612 that weave relative to one another to form a braided (e.g., woven) sleeve around the outer coil. The outer coil and braided sleeve assembly 614 can then extend to a reel 616.

FIG. 13 illustrates a second portion 620 of the manufacturing arrangement. In second portion 620, the outer coil and braided sleeve assembly 614 extends from reel 616 to extrusion mold 624. Extrusion mold 624 is arranged to continuously extrude an outer sheath around the assembly 614 as the assembly is advanced therethrough. The outer sheath assembly 626 extends from the extrusion mold 624 to a finished goods reel 630.

While one manufacturing arrangement has been illustrated and described, the present disclosure is not limited to such. For example, reel 616 may be omitted and the outer coil and braided sleeve assembly 614 may extend directly from fixture 610 to the extrusion mold 624.

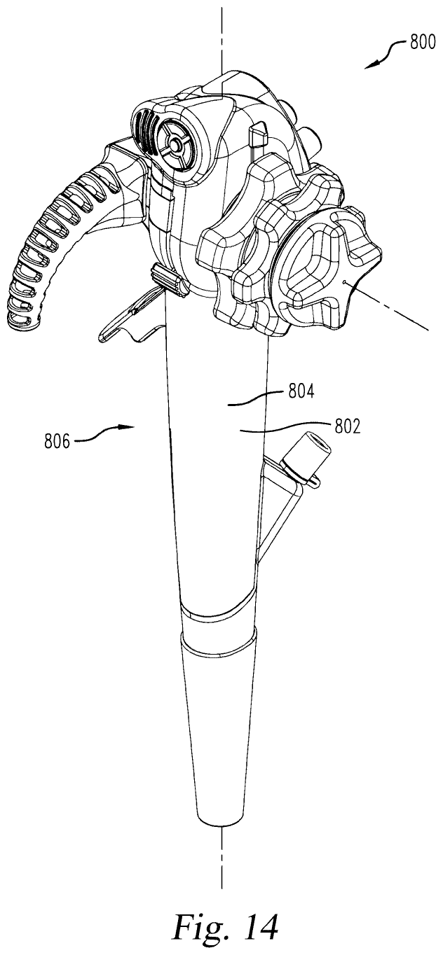

Turning to FIGS. 14-21, another exemplary example of an insertion tube 730 is illustrated. It is understood that the examples shown in FIGS. 14-21 include many of the same characteristics as the embodiments shown in FIGS. 1-13. Therefore, for the sake of readability, all of the disclosure relating to FIGS. 1-13 are incorporated herein in relation to FIGS. 14-21.

The disclosure relates to insertion tubes with static graded stiffness along the length. The disclosure, as discussed in more detail below, can include a uniform (along the length) flexible insertion tube that can be made in a low-cost fashion by continuous processes, including continuous coiling, continuous braiding, and continuous over-extrusion of a polymeric jacket. The composite insertion tube assembly may have a uniform stiffness along its length. It may also contain a stiffening element fabricated from fiberglass and resin. That stiffening element can terminates prior to the distal end of the insertion tube to achieve a fixed but graded stiffness along the length of the insertion tube.

FIG. 14 illustrates an endoscope assembly 800. The endoscope assembly includes an endoscope handle 802 made of two components. FIG. 14 illustrates the endoscope assembly with a reusable hand-piece 804 attached to a single-use shaft assembly 806.

The reusable hand-piece is selectively attachable and detachable from the single-use shaft assembly such that the reusable hand-piece may be used serially with a number of single-use shaft assemblies while the single-use shaft assembly can be discarded and/or reconditioned after a single use

The single-use shaft assembly can include an insertion tube 730 for insertion within the body of a patient.

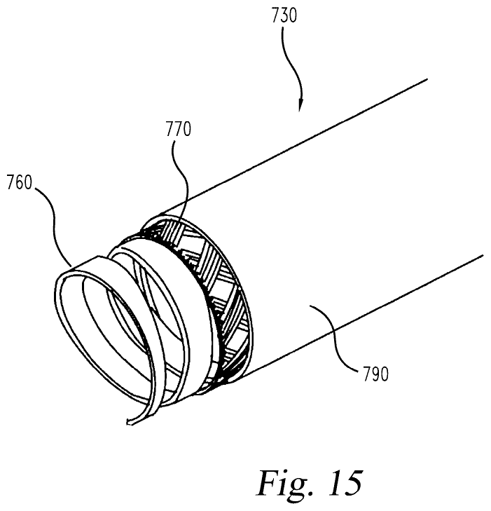

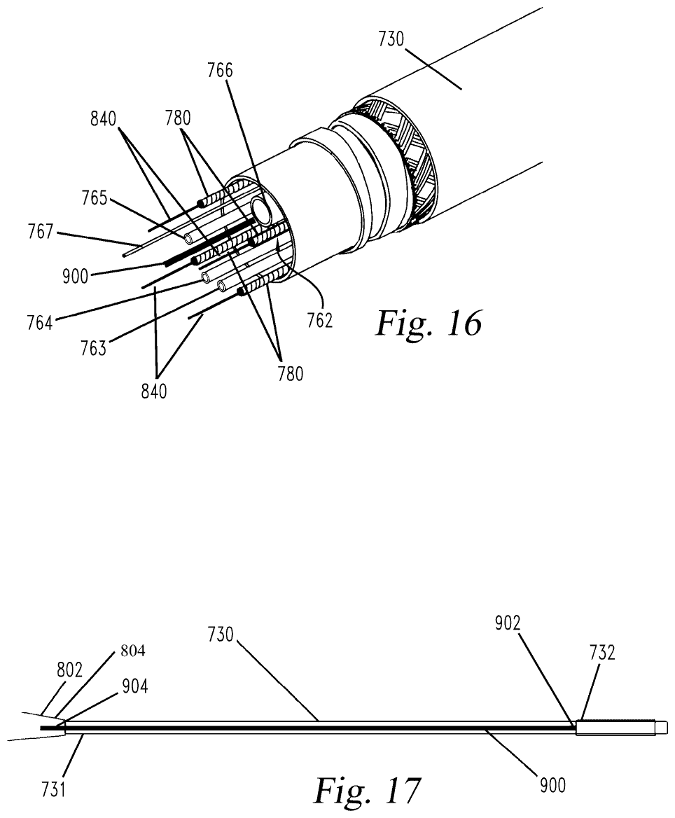

Looking to FIGS. 15 and 16, the insertion tube 730 includes an outer coil 760. An articulation wire 840 and a compression coil 780 may extend along a length of outer coil through its interior.

A braided sleeve 770 may surround the outer coil and may be positioned between the outer coil and an outer sheath 790. The outer sheath may be be applied over the braided sleeve and outer coil as a reflowed tube (e.g., thermal lamination) or by extrusion.

The outer coil may define a lumen 762 extending through the interior of the insertion tube. Various tubing and electrical components may be extended through the insertion tube to pass various fluids, tools and signals between a distal end of the insertion tube and the proximal end of the insertion tube to and from the handle, a console and/or a monitor. For example, as illustrated in FIG. 16, an air extrusion/tube 763, a camera flush extrusion/tube 764, an irrigation extrusion/tube 765, a tool channel extrusion/lumen 766, a wire bundle 767 for electrically connecting an LED or camera at the distal end of the insertion tube to the endoscope handle, console, and/or monitor, articulation wires 840, compression coils 780, and/or a stiffening element 900 may extend at least partially through the insertion tube.

The stiffening element may include a distal end 902 and a proximal end 904. The stiffening element may be inserted into the insertion tube to create variable stiffness along either substantially the entire length or a portion of the length of the insertion tube. The proximal end of the stiffening element may be fixed to a handle body of the single-use shaft assembly 806 that connects to the reusable hand-piece 804 and/or a proximal end 731 of the insertion tube. In some examples, a pocket is used to retain the proximal end of the stiffening element within the single-use shaft assembly 804. The stiffening element may be affixed at one or more locations along the length of the single-use shaft assembly. In some examples, the proximal end of the stiffening element is fixed midway along the length of the single-use shaft assembly. The stiffening element may be retained in a number of ways, for example secured within a pocket that retains the proximal end of the stiffening element via a friction fit, an interference fit, retention geometry, and/or an adhesive. For example, the proximal end of the stiffening element may be affixed to the single-use shaft assembly using an adhesive.

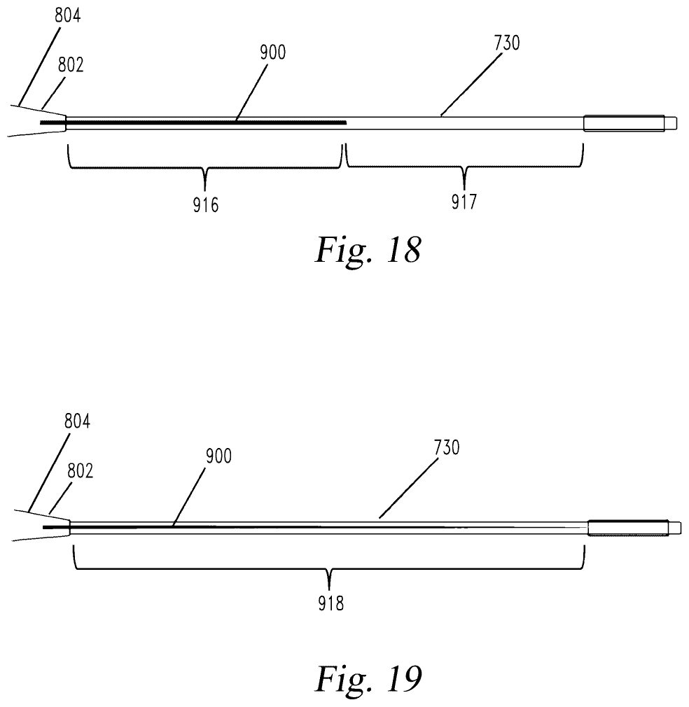

FIG. 17 illustrates the stiffening element extending from the endoscope handle to a distal end 732 of the insertion tube where it connects to an articulating link assembly 50. FIG. 18 illustrates the stiffening element extending from the endoscope handle along a portion of the length of the insertion tube. In various embodiments, the stiffener element may extend along various lengths of the insertion tube. In some examples, the stiffener element may extend from a proximal, central or any locations within the insertion tube rather than from the endoscope handle. Preferably, the stiffening element terminates short of the articulating link assembly. The articulating link assembly may include an over molded rubber over the links as shown to the right of the distal end 732 in FIGS. 17-19 and 21.

In some examples, the distal end of the stiffening element is free to move (e.g., slide and/or rotate) within the insertion tube.

In other embodiments, the distal end of the stiffening element is fixed within the insertion tube along the insertion tube length. For example, the distal end of the stiffening element may be fixed in position at the distal end of the insertion tube.

The stiffening element may be composed of a uniform material with the same modulus of elasticity along substantially the entire length of the stiffening element. In some embodiments, where the stiffening element does not extend along substantially the entire length of the insertion tube, this creates a graded stiffness along the length of the insertion tube. One stiffness for a first portion 916 with the stiffening element and a second stiffness for a second portion 917 without the stiffening element.

Turning to FIG. 19, a cross-section of the insertion tube with a tapered stiffening element 918 is shown. This creates a graded stiffness along a length of the insertion tube.

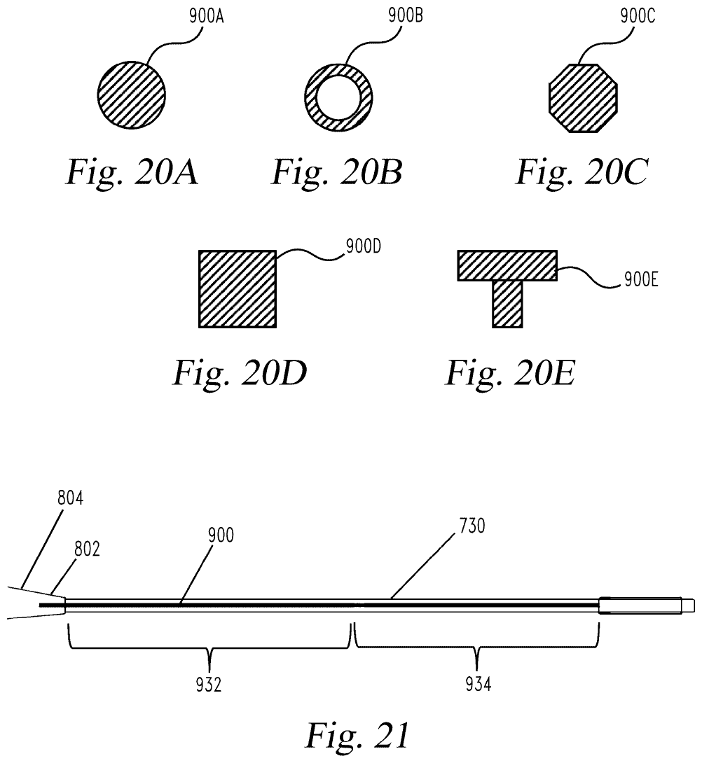

FIGS. 20A-E illustrate examples of various cross-sectional geometry options for the stiffening element. In some embodiments, one geometry may be used along a first length of the stiffening element and a second, different geometry may be used along a second length of the stiffening element. This arrangement creates a graded stiffness along the length of the insertion tube. In some examples, three or more different cross-sectional geometries may be used along the length of the stiffening element.

In other embodiments, an asymmetric cross-sectional geometry may be used to create a bias to bend in a specific direction.

Further, varying asymmetric cross-sectional geometry may be used along the length of the stiffening device to alter which direction the insertion tube is biased to turn at different points along the length of the insertion tube.

Turning to FIG. 21, an insertion tube with a stiffening element composed of uniform cross-sectional geometry is shown, but one or more materials of differing modulus of elasticities are used along the length of the stiffening element. In other words, along a first length 932 of the stiffening element, a first material with a first elasticity is used. Along a second length 934 of the stiffening element, a second material with a second elasticity is used. The second elasticity being different from the first. In some examples, three or more materials with different elasticities may be used along the length of the stiffening element.

In some examples, the stiffening element is comprised of a uniform material with the same modulus of elasticity and cross-sectional geometry along substantially its entire length, but the stiffening element terminates prior to the distal end of the insertion tube to achieve a fixed but graded stiffness along the length of the insertion tube.

In other examples, the stiffening element is comprised of a uniform material with the same modulus of elasticity along its length, but the cross-sectional geometry (see FIGS. 20A-E) is varied along its length to achieve a fixed but graded stiffness along the length of the insertion tube.

In yet other examples, the stiffening element is comprised of a uniform material with the same modulus of elasticity along its length, but the cross-sectional geometry is asymmetric about its cross-section. This creates a biased bending modulus in different directions along the length of the insertion tube.

In other embodiments, the stiffening element is comprised of a uniform cross-sectional geometry with one or more materials of differing modulus of elasticities used along the length of the stiffening element to achieve a fixed but graded stiffness along the length of the insertion tube.

In some embodiments, the stiffening element may be removeable from the insertion tube. In these embodiments, stiffening elements with various different characteristics, as detailed above, may be used in the same insertion tube depending on the patient and the procedure being performed.

After the method described in relation to FIG. 11, the stiffening element may be inserted into the insertion tube before or after cutting the insertion tube to length. The proximal end of the stiffening element may be fixed to the single-use shaft assembly after at least a portion of the stiffening element is inserted into the insertion tube.

As will be appreciated by those skilled in the art, this continuous technique for fabricating the insertion tube avoids labor and time intensive batch processes currently used to form discrete sections of endoscope shafts. Applicant believes this can reduce the costs of manufacturing endoscope shaft assemblies and/or increase production speed.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

### CLAUSES

The following numbered clauses set out specific embodiments that may be useful in understanding the present disclosure:

Clause 1: A method of fabricating a flexible endoscope shaft, comprising: a proximal insertion tube and a distal articulating section, wherein proximal insertion tube is comprised of two or more pull wires, each located within a concentric compression coil, an outer coil, an outer braid, and an outer smooth sheath, wherein the outer coil is created using a continuous process and the outer braid is applied using discrete wires in a continuous process.

Clause 2: The method of clause 1 further comprises advancing the braided assembly through an opening in a die of an extrusion mold so as to continuously apply an outer sheath to the braided assembly in a reel to reel process.

Clause 3: The method of clause 2 further comprising two or more extruders feeding resin into the extrusion die, wherein the multiple extruders apply resins of different durometers, wherein the stiffness of the shaft can be varied by applying different durometer resins, wherein a marker or unique identifier is applied to the shaft that identifies the cut location, wherein the cut shaft provided the desired stiffness in two or more segments of the shaft.

Clause 4: A method of fabricating at least a portion of a flexible endoscope shaft, comprising: forming a continuous first length, second length, and third length of outer coil using a continuous wire coiler, wherein forming the first length defines a first period of time, forming the second length defines a second period of time, and forming the third length defines a third period of time; and forming an outer braid around said outer coil along the first length during said second period of time to make a braided assembly.

Clause 5: The method of clause 4, comprising: positioning within said outer coil two or more pull wires, each pull wire located within a concentric compression coil.

Clause 6: A method of any preceding clause, wherein the flexible endoscope shaft comprises a distal articulating section extending distally of the proximal insertion tube.

Clause 7: The method of any preceding clause, wherein the outer braid is formed with discrete wires.

Clause 8: The method of any preceding clause, comprising advancing the braided assembly through an opening in a die of an extrusion mold during said third period of time.

Clause 9: The method of any preceding clause, comprising forming an outer braid around said outer coil along the second length during said third period of time.

Clause 10: The method of any preceding clause further comprising applying resins of different durometers along said first length.

Clause 11: The method of any preceding clause comprising: applying an identifier to the assembly identifying a cut location.

Clause 12: The method of any preceding clause, wherein a length of the assembly having resins of different durometers is free of the identifier.

Clause 13: A method of fabricating at least a portion of an insertion tube, comprising: forming a continuous first length, second length, and third length of outer coil using a continuous wire coiler, wherein forming the first length defines a first period of time, forming the second length defines a second period of time, and forming the third length defines a third period of time; forming an outer braid around said outer coil along the first length during said second period of time to make a braided assembly; affixing a proximal end of a stiffening element to a single-use shaft assembly of an endoscope; and inserting the stiffening element within the insertion tube along at least a portion of a length of the insertion tube.

Clause 14: The method of any preceding clause, wherein the stiffening element extends along substantially the entire length of the insertion tube after inserting the stiffening element within the insertion tube.

Clause 15: The method of any preceding clause, comprising affixing the stiffening element to a portion of an endoscope handle with an adhesive.

Clause 16: The method of any preceding clause, comprising affixing the proximal end of the stiffening element to a proximal end of the insertion tube.

Clause 17: The method of any preceding clause, wherein a distal end of the stiffening element is freely moveable within the insertion tube after inserting the stiffening element within the insertion tube.

Clause 18: The method of any preceding clause, comprising affixing a distal end of the stiffening element along a portion of a length of the insertion tube after inserting the stiffening element within the insertion tube.

Clause 19: The method of any preceding clause, comprising affixing a distal end of the stiffening element to a distal portion of the insertion tube.

Clause 20: The method of any preceding clause, wherein the stiffening element is comprised of a uniform material with the same elasticity and cross-sectional geometry along substantially the entire length of the stiffening element, wherein inserting the stiffening element extends the stiffening element along a length of the insertion tube, and wherein after said inserting the stiffening element terminates prior to a distal end of the insertion tube.

Clause 21: The method of any preceding clause, wherein the stiffening element includes a first length and a second length, wherein the stiffening element is comprised of a uniform material with the same elasticity along the first length and the second length of the stiffening element, wherein the stiffening element includes a first cross-sectional geometry along the first length and a second cross-sectional geometry along the second length, and wherein the first cross-sectional geometry is different than the second-cross-sectional geometry.

Clause 22: The method of any preceding clause, wherein the stiffening element is comprised of a uniform material with the same elasticity and cross-sectional geometry along substantially the entire length of the stiffening element, and wherein the cross-sectional geometry is asymmetrical.

Clause 23: The method of any preceding clause, wherein the stiffening element includes a first length and a second length, wherein the first length is comprised of a first material and the second length is comprised of a second material, and wherein the first material has a different modulus of elasticity than a modulus of elasticity of the second material.

Clause 24: A single-use shaft assembly of an endoscope assembly, comprising: an outer coil with a length extending along at least a portion of the length of the insertion tube; a braided sleeve surrounding at least a portion of the length of the outer coil; an outer sheath surround the braided sleeve and outer coil; a stiffening element extending along at least a portion of the length of the insertion tube, wherein a proximal end of the stiffening element is affixed to a housing of the single-use shaft assembly.

Clause 25: The single-use shaft assembly of clause 24, wherein the stiffening element extends along substantially the entire length of the insertion tube.

Clause 26: The single-use shaft assembly of any one of clauses 24-25, wherein the stiffening element is fixed to a portion of a housing of the single-use shaft assembly using an adhesive.

Clause 27: The single-use shaft assembly of any one of clauses 24-26, wherein the stiffening element is fixed to a proximal end of the insertion tube.

Clause 28: The single-use shaft assembly of any one of clauses 24-27, wherein a distal end of the stiffening element is freely moveable within the insertion tube.

Clause 29: The single-use shaft assembly of any one of clauses 24-28, wherein a distal end of the stiffening element is fixed along a portion of a length of the insertion tube.

Clause 30: The single-use shaft assembly of any one of clauses 24-29, wherein a distal end of the stiffening element is fixed to a distal portion of the insertion tube.

Clause 31: The single-use shaft assembly of any one of clauses 24-30, wherein the stiffening element is comprised of a uniform material with the same elasticity and cross-sectional geometry along substantially the entire length of the stiffening element, wherein the stiffening element extends along a length of the insertion tube, and wherein the stiffening element terminates prior to a distal end of the insertion tube.

Clause 32: The single-use shaft assembly of any one of clauses 24-31, wherein the stiffening element includes a first length and a second length, wherein the stiffening element is comprised of a uniform material with the same elasticity along the first length and the second length of the stiffening element, wherein the stiffening includes a first cross-sectional geometry along the first length and a second cross-sectional geometry along the second length, and wherein the first cross-sectional geometry is different than the second-cross-sectional geometry.

Clause 33: The single-use shaft assembly of any one of clauses 24-32, wherein the stiffening element is comprised of a uniform material with the same elasticity and cross-sectional geometry along substantially the entire length of the stiffening element, and wherein the cross-sectional geometry is asymmetrical.

Clause 34: The single-use shaft assembly of any one of clauses 24-33, wherein the stiffening element includes a first length and a second length, wherein the first length is comprised of a first material and the second length is comprised of a second material, and wherein the first material has a different modulus of elasticity than a modulus of elasticity of the second material.

1. A method of fabricating at least a portion of an insertion tube, comprising:
forming a continuous first length, second length, and third length of outer coil using a continuous wire coiler, wherein forming the first length defines a first period of time, forming the second length defines a second period of time, and forming the third length defines a third period of time;
forming an outer braid around said outer coil along the first length during said second period of time to make a braided assembly;
affixing a proximal end of a stiffening element to a single-use shaft assembly of an endoscope; and
inserting the stiffening element within the insertion tube along at least a portion of a length of the insertion tube.

2. The method of claim 1, wherein the stiffening element extends along substantially the entire length of the insertion tube after inserting the stiffening element within the insertion tube.

3. The method of claim 1, comprising affixing the stiffening element to a portion of a housing of the single-use shaft assembly with an adhesive.

4. The method of claim 1, comprising affixing the proximal end of the stiffening element to a proximal end of the insertion tube.

5. The method of claim 1, wherein a distal end of the stiffening element is freely moveable within the insertion tube after inserting the stiffening element within the insertion tube.

6. The method of claim 1, comprising affixing a distal end of the stiffening element a portion of a length of the insertion tube after inserting the stiffening element within the insertion tube.

7. The method of claim 1, comprising affixing a distal end of the stiffening element to a distal portion of the insertion tube.

8. The method of claim 1, wherein the stiffening element is comprised of a uniform material with the same elasticity and cross-sectional geometry along substantially the entire length of the stiffening element;
wherein inserting the stiffening element extends the stiffening element along a length of the insertion tube; and
wherein after said inserting the stiffening element terminates prior to a distal end of the insertion tube.

9. The method of claim 1, wherein the stiffening element includes a first length and a second length, wherein the stiffening element is comprised of a uniform material with the same elasticity along the first length and the second length of the stiffening element, wherein the stiffening element includes a first cross-sectional geometry along the first length and a second cross-sectional geometry along the second length, and wherein the first cross-sectional geometry is different than the second-cross-sectional geometry.

10. The method of claim 1, wherein the stiffening element is comprised of a uniform material with the same elasticity and cross-sectional geometry along substantially the entire length of the stiffening element, and wherein the cross-sectional geometry is asymmetrical.

11. The method of claim 1, wherein the stiffening element includes a first length and a second length, wherein the first length is comprised of a first material and the second length is comprised of a second material, and wherein the first material has a different modulus of elasticity than a modulus of elasticity of the second material.

12. A single-use shaft assembly of an endoscope assembly, comprising:
an outer coil with a length extending along at least a portion of a length of an insertion tube;
a braided sleeve surrounding at least a portion of the length of the outer coil;
an outer sheath surround the braided sleeve and outer coil; and
a stiffening element extending along at least a portion of the length of the insertion tube, wherein a proximal end of the stiffening element is affixed to a housing of the single-use shaft assembly.

13. The single-use shaft assembly of claim 12, wherein the stiffening element extends along substantially the entire length of the insertion tube.

14. The single-use shaft assembly of claim 12, wherein the stiffening element is fixed to a portion of a housing of the single-use shaft assembly using an adhesive.

15. The single-use shaft assembly of claim 12, wherein the stiffening element is fixed to a proximal end of the insertion tube.

16. The single-use shaft assembly of claim 12, wherein a distal end of the stiffening element is freely moveable within the insertion tube.

17. The single-use shaft assembly of claim 12, wherein a distal end of the stiffening element is fixed along a portion of a length of the insertion tube.

18. The single-use shaft assembly of claim 12, wherein a distal end of the stiffening element is fixed to a distal portion of the insertion tube.

19. The single-use shaft assembly of claim 12, wherein the stiffening element is comprised of a uniform material with the same elasticity and cross-sectional geometry along substantially the entire length of the stiffening element, wherein the stiffening element extends along a length of the insertion tube, and wherein the stiffening element terminates prior to a distal end of the insertion tube.

20. The single-use shaft assembly of claim 12, wherein the stiffening element includes a first length and a second length, wherein the stiffening element is comprised of a uniform material with the same elasticity along the first length and the second length of the stiffening element, wherein the stiffening includes a first cross-sectional geometry along the first length and a second cross-sectional geometry along the second length, and wherein the first cross-sectional geometry is different than the second-cross-sectional geometry.

21. The single-use shaft assembly of claim 12, wherein the stiffening element is comprised of a uniform material with the same elasticity and cross-sectional geometry along substantially the entire length of the stiffening element, and wherein the cross-sectional geometry is asymmetrical.

22. The single-use shaft assembly of claim 12, wherein the stiffening element includes a first length and a second length, wherein the first length is comprised of a first material and the second length is comprised of a second material, and wherein the first material has a different modulus of elasticity than a modulus elasticity of the second material.
